(19) United States
(12) Patent Application Publication
(10) Pub. No.: US 2021/0255181 A1
(43) Pub. Date: Aug. 19, 2021

(54) DIAGNOSTIC ASSAYS USING BACTERIAL QUORUM SENSING SIGNALS

(71) Applicant: Quorum X Diagnostics, Inc., Avondale Estates, GA (US)

(72) Inventors: Maria Margaret Nagy, Avondale Estates, GA (US); Angel A. Rivera, Decatur, GA (US)

(21) Appl. No.: 17/253,436

(22) PCT Filed: Jun. 24, 2019
(86) PCT No.: PCT/US2019/038735

§ 371 Date: Dec. 17, 2020

Related U.S. Application Data
(60) Provisional application No. 62/689,072, filed on Jun. 23, 2018.

(51) Int. Cl. G01N 33/558; G01N 33/569

(52) U.S. Cl. CPC G01N 33/558; G01N 2333/21; G01N 33/56911

(57) ABSTRACT

Disclosed are methods, systems, devices, and kits for diagnosing and/or monitoring a bacterial infection in a subject in need thereof. The methods, systems, devices, and kits are useful for identifying causative bacteria, such as Pseudomonas aeruginosa, by, for example, detecting the presence of one or more quorum sensing (QS) molecules associated with the bacteria. For P. aeruginosa these can include C4 homoserine lactone and/or C12 homoserine lactone. The methods, systems, devices, and kits can further include steps or features for obtaining diagnosis results and/or prescribing an antibiotic for the subject if, for example, the presence and/or increased levels of the QS molecule are detected. The methods, systems, devices, and kits can further include steps or features guiding a treatment regimen, such as whether to initiate or continue treatment of a bacterial infection caused by bacteria, such as P. aeruginosa, detected using the disclosed methods, systems, devices, and kits.

### CROSS-REFERENCE TO RELATED APPLICATIONS

This application claims the benefit of and priority to U.S. Provisional Application No. 62/689,072 filed Jun. 23, 2018, which is hereby incorporated by reference in its entirety.

### FIELD OF THE INVENTION

The field of the invention relates to methods and kits suitable for diagnosing and monitoring bacterial infections, in particular, those caused by Pseudomonas aeruginosa or related organism.

### BACKGROUND OF THE INVENTION

According to the CDC, there are 23,000 deaths in the US alone caused by multidrug resistant bacterial infections and 700,000 deaths worldwide. This rate is increasing exponentially due to increased bacterial resistance. The infectious Disease Society of America (IDSA) projects that by 2030 we will be in a post antibiotic era with as many as 10,000,000 deaths per year. According to the World Health Organization (WHO), part of the reason for this it that 50% of patients have been inappropriately prescribed antibiotics. This has contributed to the increase in antibiotic resistance, which reduces the availability of effective drugs, increases secondary infections, extends hospital stays, increases lost days of work, and increases insurance premiums, among other effects.

The problem is so serious that the Obama administration put out a plea in 2015 to drug companies, academic institutions, and researchers to develop both new antibiotics and diagnostic tools. First, a S20 million dollar Combating Antibiotic Resistance Bacteria (CARB-X) challenge was put into effect in order to develop new innovative diagnostic screening methods. Second, the Centers for Medicare and Medicaid (CME) passed a mandate in June 2016 requiring all agencies that dispense antibiotics (hospitals, long term care, urgent care and doctors' offices) to implement an antibiotic stewardship program. As part of this program, quick turnaround diagnostics are essential.

The current state of antibiotic susceptibility testing (typically culture tests) can take 24-48 hours to determine the causative agent of an infection. Other methods, such as PCR and DNA electrophoresis, extract bacterial DNA and amplify it to determine if, and identify which, antibiotic resistance genes are present. While these methods are much faster (˜6 hours), the cost is extremely high and they require highly trained staff and access to expensive equipment.

In the case of pneumonia, healthcare practitioners typically prescribe broad spectrum antibiotics immediately and then wait for test results to come in reevaluate 48 hours later, the so-called “48-hour time out” before culture results are available. At that point, the patient's antibiotics will be readjusted as needed. While this re-adjustment allows the patient to be put on the correct antibiotics, it means the patient maybe on an incorrect antibiotic initially. Practices like these increase the probability of bacterial drug resistance.

Therefore, it is an object of the present invention to provide methods, systems, devices, and kits for low-cost, simple, rapid, and/or accurate detection of a bacterial infection, such as those associated with pneumonia, bronchitis, and urinary tract infections.

It is a further object of the present invention to provide methods, systems, devices, and kits for identifying causative agents of a bacterial infection, such as Pseudomonas aeruginosa.

It is a further object of the present invention to provide methods, systems, devices, and kits to better guide treatment regimens of bacterial infections, thus, for example, reducing the possibility of prescribing incorrect antibiotics.

### SUMMARY OF THE INVENTION

Disclosed are methods, systems, devices, and kits for diagnosing and/or monitoring a bacterial infection in a subject in need thereof are provided. The methods, systems, devices, and kits are useful for identifying causative bacteria, such as Pseudomonas aeruginosa, by, for example, detecting the presence of one or more quorum sensing (QS) molecules associated with the bacteria. For P. aeruginosa these can include C4 homoserine lactone and/or C12 homoserine lactone. The methods, systems, devices, and kits can further include steps or features for obtaining diagnosis results and/or prescribing an antibiotic for the subject if, for example, the presence and/or increased levels of the QS molecule are detected. The methods, systems, devices, and kits can further include steps or features guiding a treatment regimen, such as whether to initiate or continue treatment of a bacterial infection caused by bacteria, such as P. aeruginosa, detected using the disclosed methods, systems, devices, and kits.

Disclosed are methods, systems, devices, and kits detecting quorum sensing molecules. Generally, such detection is useful for identifying bacteria and fungi involved in infection of a subject, and, in particular, detection of significant infections. The methods generally use the disclosed systems, devices, and kits. In some forms, the system, device, or kit can comprise a solid support, wherein a first antibody is immobilized on the solid support, and a detection agent, wherein the detection agent comprises a detection element. In some forms, either (a) the detection agent further comprises a second antibody specific for the quorum sensing molecule and the first antibody is specific for the second antibody or (b) the detection agent further comprises the quorum sensing molecule and the first antibody is specific for the quorum sensing molecule.

In some forms of the methods, systems, devices, or kits, the solid support can be in the form of a test strip. In some forms of the systems, devices, or kits, the test strip can be an immunochromatographic test strip. In some forms of the methods, systems, devices, or kits, the test strip can comprise a conjugate pad and a membrane, wherein the membrane comprises a test line and a control line, wherein the test line is closer to the conjugate pad than the control line.

In some forms of the methods, systems, devices, or kits, the detection agent can comprise the detection element and the second antibody, wherein the first antibody is specific for the second antibody, wherein the first antibody is immobilized on the membrane at the control line, wherein the quorum sensing molecule is immobilized on the membrane at the test line, wherein the detection agent is detachably fixed on the conjugate pad.

In some forms of the methods, systems, devices, or kits, the detection agent can comprise the detection element and the quorum sensing molecule, wherein the first antibody is specific for the quorum sensing molecule, wherein the first antibody is immobilized on the membrane at both the control line and the test line, wherein the detection agent is detachably fixed on the conjugate pad.

In some forms of the methods, systems, devices, or kits, the detection agent can comprise the detection element and the second antibody, wherein the first antibody is specific for the second antibody, wherein the first antibody is immobilized on the membrane at the control line, wherein a third antibody specific for the quorum sensing molecule is immobilized on the membrane at the test line, wherein the second antibody and the third antibody bind to the quorum sensing molecule noncompetitively, wherein the detection agent is detachably fixed on the conjugate pad.

In some forms of the methods, systems, devices, or kits, the membrane can further comprise an analyte line, wherein the analyte line is disposed between the test line and the control line, wherein the quorum sensing molecule is immobilized on the membrane at the analyte line.

In some forms, the systems, devices, or kits can further comprise a reporter agent, wherein the reporter agent can facilitate detection of the detection element. In some forms of the systems, devices, or kits, the second antibody, the reporter agent, and the detection element are components of an enzyme-linked immunosorbent assay (ELISA) system.

In some forms of the methods, systems, devices, or kits, the detection element can be an enzyme, wherein the enzyme catalyzes a reaction that can produce a detectable signal. In some forms of the methods, systems, devices, or kits, the reporter agent can be an enzymatic substrate for the enzyme, wherein the enzyme can act on the reporter agent to produce the detectable signal.

Also disclosed are methods of detecting quorum sensing molecules in a sample. In some forms, the methods can detect the existence of a significant bacterial infection in a subject, the identity or classification of the organism(s) involved in the infection, or the progress of treatment of an infection.

In some forms, the methods comprise bringing into contact a sample from the subject and the solid support of any of the disclosed systems, devices, or kits and detecting the detection element on the solid support. In some forms, the presence or absence of the detection element on the solid support or at a particular location on the solid support indicates the presence of the quorum sensing molecule in the sample, wherein the indication of the presence of the quorum sensing molecule in the sample indicates the existence of a significant bacterial infection in the subject.

In some forms, the solid support is in the form of a test strip. In some forms, the test strip is an immunochromatographic test strip. In some forms, the test strip comprises a conjugate pad and a membrane, wherein the membrane comprises a test line and a control line, wherein the test line is closer to the conjugate pad than the control line. In some forms, the detection agent comprises the detection element and the second antibody, wherein the first antibody is specific for the second antibody, wherein the first antibody is immobilized on the membrane at the control line, wherein the quorum sensing molecule is immobilized on the membrane at the test line, wherein the detection agent is detachably fixed on the conjugate pad. In some forms, the absence of the detection element at test line on the solid support indicates the presence of the quorum sensing molecule in the sample.

In some forms, the detection agent comprises the detection element and the quorum sensing molecule, wherein the first antibody is specific for the quorum sensing molecule, wherein the first antibody is immobilized on the membrane at both the control line and the test line, wherein the detection agent is detachably fixed on the conjugate pad. In some forms, the absence of the detection element at test line on the solid support indicates the presence of the quorum sensing molecule in the sample.

In some forms, the detection agent comprises the detection element and the second antibody, wherein the first antibody is specific for the second antibody, wherein the first antibody is immobilized on the membrane at the control line, wherein a third antibody specific for the quorum sensing molecule is immobilized on the membrane at the test line, wherein the second antibody and the third antibody bind to the quorum sensing molecule noncompetitively, wherein the detection agent is detachably fixed on the conjugate pad. In some forms, the membrane further comprises an analyte line, wherein the analyte line is disposed between the test line and the control line, wherein the quorum sensing molecule is immobilized on the membrane at the analyte line. In some forms, the presence of the detection element at test line on the solid support indicates the presence of the quorum sensing molecule in the sample.

Also disclosed are diagnostic tools for elucidation of infectious diseases by identification of bacteria and fungi. The disclosed systems, devices, and kits are examples of such diagnostic tools. In some forms, the diagnostic tool uses a characterized detection molecule and at least one detecting field. In some forms, target molecules for the diagnostic tool can be C12-BSA, C12 HCL, C4 HCL, and associated antibodies of these group. In some forms, the diagnostic tool can be used for gram positive quorum sensing and for gram negative quorum sensing.

Also disclosed are methods of using the diagnostic tools using sputum, blood, urine, swabs, and other respiratory samples, such as tracheal, bronchoalveolar, and bronchial washes or lavage. In some forms, the methods use Lateral Flow, TLC, HPLC, or ELISA.

In some forms, the infectious diseases are defined as causative of specific transmittable illness that are originated by either bacteria or fungi. In some forms, the infectious diseases include, but are not limited to, respiratory ailments such as pneumonia, UTI, bronchitis, wounds, abscess, and others. In some forms, the diagnostic tool can be used to define bacteria and fungi, optionally defining gram morphology, gram stain for bacterial and fungus genus and or species. In some forms, the diagnostic tool can be used to further characterize bacteria and fungi by detecting and/or distinguishing colonialization vs infection, quantifying vs qualifying, multi microbe infections, colony or poly-colonial or multiple species infection (how many types of bacteria reside in the infection).

In some forms, the diagnostic tool can be used quorum sensing molecules as a detecting moiety. In some forms, the diagnostic tool can be used for gram positive bacteria, using oligo peptides for detection. In some forms, the diagnostic tool can be used for gram negative bacteria existence, using quorum sensing molecule N-Acyl homoserine lactone for identification. In some forms, the diagnostic tool can be used in relation to fungi infection, using quorum sensing auto inducers for detection.

Also disclosed are methods of developing of diagnostic tools for bacterial species identification. Also disclosed are methods for detection of virulent bacterial species using quorum sensing molecules. Also disclosed are methods using the disclosed diagnostic tools and using samples from sputum, urine or other body fluids. Also disclosed are methods to measure inhibition signal. Also disclosed are methods to qualify signal. Also disclosed are methods to quantify quorum signal either using antibodies or the signal directly. Also disclosed are methods to characterize the bacterial genus or species. Also disclosed are methods for diagnosis of pneumonia, UTI, wound infections, among others.

### BRIEF DESCRIPTION OF THE DRAWINGS

FIG. 1 is a diagram showing typical configuration of a lateral flow immunoassay test strip for the recognition of one or more analytes such as one or more quorum sensing molecules. Lateral flow test strip is typically composed of the following elements: sample pad, conjugate release pad, membrane with immobilized antibodies, and adsorbent pad. The components of the strip are usually fixed to an inert backing material. Here, a simple paper-based device is shown to distinguish the presence (or absence) of a target analyte in liquid sample (matrix) without the need for specialized and expensive equipment.

### DETAILED DESCRIPTION OF THE INVENTION

Although immunoassays are known, the effectiveness and sensitivity of these assays can vary depending on the targets, format, and materials used in the immunoassay. For example, the effectiveness and sensitivity of a given immunoassay of a given format to detect a given analyte or type of analyte is demonstrated by the targets, format, and materials used in the immunoassay and can be greater than expected or greater than is typical.

It was discovered that the sensitivity of a lateral flow assay for detection of quorum sensing molecules was greater than they expected and that detection of the quorum sensing molecule of P. aeruginosa from sputum was surprising given the low concentration of the molecules. It is believed that this result was due to the surprising sensitivity of the lateral flow assay. The sensitivity was surprising because other assay modes (e.g., dot blot) suggested that the lateral flow assay would not be as sensitive.

### I. Definitions

The singular forms “a,” “an,” and “the” include plural reference unless the context clearly dictates otherwise. For example, reference to “a compound” includes a plurality of compounds and reference to “the compound” is a reference to one or more compounds and equivalents thereof known to those skilled in the art.

The terms “can,” and “can be,” and related terms are intended to convey that the subject matter involved is optional (that is, the subject matter is present in some forms and is not present in other forms), not a reference to a capability of the subject matter or to a probability, unless the context clearly indicates otherwise.

The term “antibiotic” or like words or other forms refers to a compound, substance, molecule, or composition, which acts to reduce, inhibit, or prevent an infection of bacteria. Exemplary antibiotics are those used to treat P. aeruginosa infections including aminoglycosides (gentamicin, tobramycin, amikacin, netilmicin), carbapenems (imipenem, meropenem), cephalosporins (ceftazidime, cefepime), fluoroquinolones (ciprofloxacin, levofloxacin), penicillin with β-lactamase inhibitors (BLI) (ticarcillin and piperacillin in combination with clavulanic acid or tazobactam), monobactams (aztreonam), fosfomycin and polymyxins (colistin, polymyxin B).

The term “assaying,” “assay,” or like terms refers to an analysis to determine a characteristic of a substance such as a molecule or an analyte, for example, the presence, absence, quantity, extent, kinetics, dynamics, or binding.

The term “analyte” is generally used to refer to any molecule or component that is to be detected or assessed. For example, a quorum sensing (QS) molecule to be detected in the disclosed methods is an analyte.

The term “antigen” refers to a molecule or component that can be bound by an antibody and/or used to raise or generate antibodies or an immune reaction. As an example, analytes that bind to or are bound by an antibody are antigens. Use herein of the term antigen should be considered to also be a reference to analyte in this context.

An “assay output” or like terms or other forms refers to the result or product from running an assay, such as data. For example, an assay output could be the fact that one or more biomarkers such as N-butanoyl-L homoserine lactone (C4-HSL) and/or N-(3-oxododecanoyl)-L-homoserine lactone (3-oxo-C12-HSL) are present in a sample, after running the assay which tested whether the biomarkers were present or not. The assay can be expressed in a readout on a screen, on a paper, or in any other media, such as a computer disk, but it must be expressed. In other words, the presence of N-butanoyl-L homoserine lactone (C4-HSL) and/or N-(3-oxododecanoyl)-L-homoserine lactone (3-oxo-C12-HSL) is not the assay output, rather, it is the expression of this fact in some tangible form that is the assay output.

The term “binding affinity” can be defined as two molecules interacting with a K_{D} of at least 10⁻³, 10⁻⁴, 10⁻⁵, 10⁻⁶, 10⁻⁷, 10⁻⁸, 10⁻⁹ 10⁻¹⁰, or 10⁻¹¹, or 10⁻¹² M or tighter binding, and can refer to for example, molecules including antibodies which bind biomarkers such as N-butanoyl-L homoserine lactone (C4-HSL) and N-(3-oxododecanoyl)-L-homoserine lactone (3-oxo-C12-HSL).

The term “complex” as used herein refers to the association of a first molecule with another molecule for which the first molecule has a binding affinity, for example, a complex formed of N-butanoyl-L homoserine lactone bound and its specific antibody.

“Contacting” or like terms means bringing into proximity such that a molecular interaction can take place, if a molecular interaction is possible between at least two things, such as molecules, cells, markers, compounds, or compositions, or any of these with an article or with a machine. For example, contacting refers to bringing at least two compositions, molecules, articles, or things into contact, i.e., such that they are in proximity to mix or touch. It is understood that anything disclosed herein can be brought into contact with anything else. For example, a sample can be brought into contact with a reagent, such as an antibody that binds C4-HSL and 3-oxo-C12-HSL, and so forth.

A “positive control” or like terms is a control that shows that the conditions for data collection can lead to data collection. In specific examples, a positive control is a sample containing a target analyte such as C4-HSL and 3-oxo-C12-HSL.

The terms “control” or “control levels” or like terms are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard. For example, a control can refer to the results from an experiment in which the subjects, objects, or reagents are treated as in a parallel experiment except for omission of the procedure, agent, or variable under test and which is used as a standard of comparison in judging experimental or assay effects. Thus, the control can be used to determine the effects related to the procedure, agent, or variable. For example, if the effect of a test molecule on a cell was in question, one could (a) simply record the characteristics of the cell in the presence of the molecule, (b) perform an assay and then also record the effects of adding a control molecule or a control composition with a known activity or lack of activity (e.g., the assay buffer solution) and then compare effects of the test molecule to the control. In certain circumstances, once a control is performed the control can be used as a standard, in which the control experiment does not have to be performed again. In other circumstances, the control experiment can or should be run in parallel each time a comparison will be made.

“Comparing” or like words or other forms refers to the act of reviewing something in relation to something else.

Confidence intervals can be provided as, for example, + or −5%, 10%, 15%, 20%, 30%, 40%, 50%, 75%, or 100%. For example, the disclosed methods and assays can determine, for example, the presence of a bacterial infection, with, for example, at least a 50%, 60%, 70%, 80%, 90%, 95% 97%, or 99% certainty.

“Determining” or like words or other forms refers to the act of settling or deciding by choice from different alternatives or possibilities.

By “inhibit” or other forms of inhibit means to hinder or restrain a particular characteristic. It is understood that this is typically in relation to some standard or expected value, in other words it is relative, but that it is not always necessary for the standard or relative value to be referred to. For example, “inhibits bacterial growth” means hindering or restraining the amount of bacterial burden relative to a standard or a control.

Normalizing or like terms means, adjusting data, or a response, or an assay result, for example, to remove at least one common variable.

“Optional” or “optionally” means that the subsequently described event or circumstance may or may not occur, and that the description includes instances where said event or circumstance occurs and instances where it does not.

The term “obtaining” or like words or other forms refers to getting or receiving or attaining. It requires to a planned effort by the actor, but the plan can be in acceptance, for example, by accepting something that is given one.

As used herein, the term “pharmacological activity” refers to the inherent physical properties of a peptide or polypeptide. These properties include but are not limited to half-life, solubility, and stability and other pharmacokinetic properties.

By “prevent” or other forms of prevent means to stop a particular characteristic or condition. Prevent does not require comparison to a control as it is typically more absolute than, for example, reduce or inhibit. As used herein, something could be reduced but not inhibited or prevented, but something that is reduced could also be inhibited or prevented. It is understood that where reduce, inhibit or prevent are used, unless specifically indicated otherwise, the use of the other two words is also expressly disclosed. Thus, if inhibition of phosphorylation is disclosed, then reduction and prevention of phosphorylation are also disclosed.

The terms “prescribing” or “prescription” or like words or other forms refers to a written direction or act for a therapeutic or corrective agent; specifically, such as one for the preparation and use of a medication.

By “sample” or like terms is meant a natural product, a natural product extract, etc.; a tissue or organ from an animal; a cell (either within a subject, taken directly from a subject; or any bodily fluid or excretion (for example, but not limited to, serum, blood, urine, stool, saliva, tears, bile), which is assayed as described herein. In particular, a sample can be sputum, blood, urine, swabs, and other respiratory samples, such as tracheal, bronchoalveolar, and bronchial washes or lavage.

As used throughout, by a “subject” is meant an individual. A subject can be a patient. A subject can be a human.

A “standard” or like terms or other forms refers to an established rule or measure that has been previously determined, but which can be used for comparative purposes. It often is used like a control, and often it is produced by running a control or multiple control experiments to determine a consistent or average result as a “control.”

The term “transmitting the assay output to a recipient” or like terms or other forms refers to the act of sending an assay output. This can refer to for example, refer to an email from a computer, automatically generated to, for example, a doctor or doctor's office.

The term “treating” or “treatment” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease. In certain situations a treatment can inadvertently cause harm.

The term “therapeutically effective” means that the amount of the composition used is of sufficient quantity to ameliorate one or more causes or symptoms of a disease or disorder. Such amelioration only requires a reduction or alteration or decrease, not necessarily elimination.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

By “reduce” or other forms of reduce means lowering of an event or characteristic. It is understood that this is typically in relation to some standard or expected value, in other words it is relative, but that it is not always necessary for the standard or relative value to be referred to. For example, “reduces bacterial infection” means lowering the amount of bacterial count/burden or alleviate one or more symptoms associated with the bacterial infection relative to a standard or a control.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data are provided in a number of different formats, and that this data, represents endpoints and starting points, and ranges for any combination of the data points. For example, if a particular datum point “10” and a particular datum point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### II. Systems

Systems for detection of analytes in biological fluids are described. In particular, systems are described that are suitable for detection of analytes including, for example, one or more quorum sensing (QS) molecules associated with particular bacteria, such as P. aeruginosa. Exemplary biological fluids include bronchial lavage, sputum, tracheal, bronchoalveolar, and bronchial washes or lavage, urine, blood and other bodily fluids. In some forms, the systems can embody, facilitate, or use immunochemical assays and reagents for qualitative or quantitative detection of analytes in biological fluids. Exemplary immunochemical assays include ELISA, dot-blot, and lateral flow. In some forms, the systems can embody, facilitate, or use analytical assays and reagents for qualitative or quantitative detection of analytes in biological fluids are described. Exemplary analytical assays include thin layer chromatography (TLC) and high-performance liquid chromatography (HPLC).

In some forms, the systems embody, facilitate, or use a device or kit as disclosed. Generally, the disclosed methods use or involve the disclosed systems, devices, kits, and combinations thereof.

Exemplary analytes to be detected by one of the immunochemical assays or analytical assays are one or more QS molecules, for example, those associated with P. aeruginosa. In some forms, the QS molecule is an N-acyl homoserine lactone (AHL) such as N-butyryl homoserine lactone (C4-HSL), N-hexanoyl homoserine lactone (C6-HSL), N-(3-oxo)-hexanoyl homoserine lactone (3-oxo-C6-HSL), N-octanoyl homoserine lactone (C8-HSL), N-(3-oxo)-octanoyl homoserine lactone (3-oxo-C8-HSL), N-decanoyl homoserine lactone (C10-HSL), N-dodecanoyl homoserine lactone (C12-HSL), N-(3-oxo)-dodecanoyl homoserine lactone (3-oxo-C12-HSL), N-tetradecanoyl homoserine lactone (C14-HSL), and combinations thereof. In some forms, the QS molecule is N-butanoyl-L homoserine lactone (C4-HSL) and/or N-(3-oxododecanoyl)-L-homoserine lactone (3-oxo-C12-HSL).

A. Immunoassays

Immunoassays can be used to detect one or more targeting QS molecules including N-acyl homoserine lactone. An immunoassay is a test that relies on biochemistry to measure the presence and/or concentration of an analyte. The analyte can be large proteins or small molecules that a person has produced as a result of an infection. These assays are highly adaptable and can be applied to many formats depending on the needs of the end user. The principle component of an immunoassay designed to detect a specific analyte, such as N-acyl homoserine lactone, is the antibodies or other molecules that have been carefully selected to ensure the detection of the analyte at low concentration with high specificity. The second feature of an immunoassay is the system that is designed to detect the binding of the specific antibody to the target analyte.

Some exemplary immunoassays suitable for detecting one or more targeting QS molecules including N-acyl homoserine lactone are described below.

Anti-antibody antibodies are useful in immunoassays. An anti-antibody antibody is an antibody that is specific for a particular antibody or class of antibodies. As useful form of anti-antibody antibodies is antibodies specific for antibody class determinants or, put another way, specific for antibodies of a particular class (such as IgG and IgM antibody classes). Because the antibody class determinants are often species-specific, it is possible and useful to us anti-antibody antibodies that are specific to antibodies from a particular species. Anti-antibody antibodies that are specific for human IgG antibodies or human IgM antibodies, for example, are useful for binding to and aiding in detection of human antibodies.

1. Lateral Flow Assays

Lateral flow assays (LFA) are also known as lateral flow immunochromatographic assays. These are simple paper-based devices intended to detect the presence (or absence) of a target analyte in liquid sample (matrix) without the need for specialized and costly equipment, though many lab-based applications exist that are supported by reading equipment. Typically, these tests are used for medical diagnostics either for home testing, point of care testing, or laboratory use. A widespread and well-known application is the home pregnancy test.

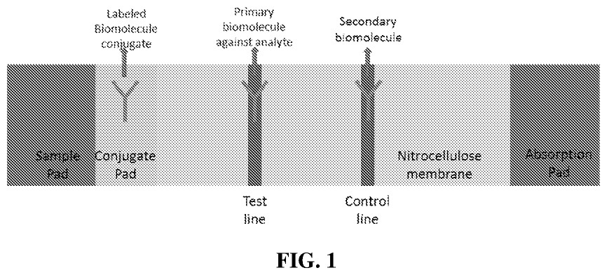

As illustrated in FIG. 1, a typical lateral flow device includes sample pad, conjugate release pad, membrane with immobilized antibodies and adsorbent pad. The components of the strip are typically fixed to an inert backing material. The principle behind LFA is simple (see review by Katarzyna M. Koczula et al., Essays Biochem. 2016 Jun. 30; 60(1): 111-120): a liquid sample (or its extract) containing the analyte of interest moves without the assistance of external forces (capillary action) through various zones of polymeric strips, on which molecules that can interact with the analyte are attached. A typical lateral flow test strip consists of overlapping pads and/or membranes that are mounted on a backing card for better stability and handling. The sample is applied at one end of the strip, on the adsorbent sample pad, which is impregnated with buffer salts and surfactants that make the sample suitable for interaction with the detection system. The sample pad ensures that the analyte present in the sample will be capable of binding to the capture reagents of conjugates and on the membrane. The treated sample migrates through the conjugate release pad, which contains antibodies or other molecules that are specific to the target analyte and are conjugated to a label (detection element), such as colored or fluorescent particles—most commonly colloidal gold and latex microspheres or enzymes. The sample, together with the conjugated antibody bound to the target analyte, migrates along the strip into the detection zone. This is a porous membrane (usually composed of nitrocellulose) with specific biological components (usually antibodies, antigens, or other molecules) immobilized in “lines.” Their role is to react with the analyte bound to the conjugated antibody. Recognition of the sample analyte results in an appropriate response on the test line, while a response on the control line indicates the proper liquid flow through the strip. The read-out, represented by the lines appearing with different intensities, can be assessed by eye or using a dedicated reader. In some formats, additional test lines of antibodies or other molecules specific to different analytes can be immobilized in an array format for detecting multiple analytes simultaneously under the same conditions. In some formats, multiple test lines loaded with the same antibody can be used for semi-quantitative assays. The principle of this ‘ladder bars’ assay is based on the stepwise capture of colorimetric conjugate-antigen complexes by the immobilized antibody on each successive line, where the number of lines appearing on the strip is directly proportional to the concentration of the analyte. The liquid flows across the device because of the capillary force of the strip material and, to maintain this movement, an absorbent pad is attached at the end of the strip. The role of the absorbent pad is to wick the excess reagents and prevent backflow of the liquid.

The “lines” for interacting with the materials in the liquid flow can be in a variety of shapes, orientations, and relationships. Most commonly, the “lines” are linear strips of material perpendicular to liquid flow. Also most commonly, different “lines” with different components are separate and do not overlap. These features are most consistent with the mechanics and operation of LFA. However, the lines can be in shapes other than a strip, can be oriented other than perpendicular to the liquid flow, and can overlap. For example, some LFA place the test line and the control line perpendicular to each other and overlapping so as to form a + symbol when both lines show a detectable signal.

Two formats of the LFA can be distinguished: direct and competitive. A direct test is used for larger analytes such as the p24 antigen used in the human immunodeficiency virus (HIV) test, as well as analytes with multiple antigenic sites such as human chorionic gonadotropin (hCG) used in pregnancy tests. The hCG test is an example of a sandwich-based assay, where the target is immobilized between two complementary antibodies. In the direct test, the presence of the test line indicates a positive result and the control line usually contains species-specific anti-immunoglobulin antibodies, specific for the antibody in the particular conjugate. In the case of small molecules with single antigenic determinants, which cannot bind to two antibodies simultaneously, competitive tests are used. In this type of test, the analyte blocks the binding sites on the antibodies on the test line, preventing their interactions with the coloured conjugate. Therefore, a positive result is indicated by the lack of signal in the test line, while the control line should be visible independently of the test result. In some forms, the lateral flow assays for detecting one or more QS molecules are competitive assays.

Lateral flow tests are widely used in human health for point of care testing. They can be performed by a healthcare professional or by the patient, and in a range of settings including the laboratory, clinic or home. They are designed to incorporate intuitive user protocols and require minimal training to operate. They can be qualitative and read visually.

Lateral flow assays are generally performed over a strip, different parts of which are assembled on a backing. The basic parts are a sample application pad, a conjugate pad, a membrane (e.g., nitrocellulose membrane), and an adsorption pad. The membrane is further divided into test and control lines. Pre-immobilized reagents at different parts of the strip become active upon flow of liquid sample. LFA combines unique advantages of analyte-binding molecules and chromatography. LFA based strips can have different detection formats.

LFA can be performed in a variety of formats. Strips used for LFA generally contain four main components. The sample application pad is generally made of cellulose and/or glass fiber and sample is applied on this pad to start assay. Its function is to transport the sample to other components of lateral flow test strip (LFTS). The sample pad should be capable of transportation of the sample in a smooth, continuous and homogenous manner Sample application pads are sometimes designed to pretreat the sample before its transportation. This pretreatment can include separation of sample components, removal of interferences, adjustment of pH, etc.

The conjugate pad is the place where labeled molecules (detection agents) that bind to the analyte (e.g., labeled antibody specific for the analyte) are dispensed. The conjugate pad should release labeled conjugate upon contact with moving liquid sample. Glass fiber, cellulose, polyesters and some other materials can be used to make the conjugate pad for LFA. The membrane is where the test and control lines are drawn. An ideal membrane should provide support and good binding to components attached or placed on the membrane. The adsorbent pad works as sink at the end of the strip. It also helps in maintaining flow rate of the liquid over the membrane and stops back flow of the sample.

All these components are generally fixed or mounted over a backing card. Materials for the backing card are highly flexible because they have nothing to do with LFA except providing a platform for proper assembly and positioning of all the components. Thus the backing card serves as a support and facilitates handling of the strip.

In a typical format, a labeled first molecule (detection agent, e.g., antibody) that binds to the analyte is prefixed at the conjugate pad. This is a temporary adsorption and the flow of buffer solution will cause the labeled first molecule to flow off of the conjugate pad. A second molecule (e.g., antibody) that binds to the analyte is immobilized over the test line. A third molecule (e.g., antibody) that binds to the conjugate of the labeled first molecule and analyte is immobilized at control zone.

A sample containing the analyte is applied to the sample application pad and it subsequently migrates to the other parts of strip. At the conjugate pad, the target analyte is captured by the prefixed labeled first molecule and results in the formation of labeled first molecule/analyte complex. This complex then flows to the membrane under capillary action. At the test line, the labeled first molecule/analyte complex is captured by the second molecule via binding to the analyte. The analyte becomes sandwiched between labeled first molecule and the second molecule, forming labeled first molecule/analyte/second molecule complex. Excess labeled first molecule/analyte complex will be captured at the control line by the third molecule. Buffer or excess solution goes to absorption pad. The intensity of color at test line corresponds to the amount of target analyte and can be measured with an optical strip reader or visually inspected. Appearance of color at control line ensures that a strip is functioning properly.

The competitive format is useful for low molecular weight compounds that cannot bind two molecules (e.g., antibody) simultaneously. In competitive format LFA, absence of color at the test line is an indication of the presence of analyte while appearance of color at both the test and control lines indicates a negative result. The competitive format has two main layouts. In the first layout, solution containing target analyte is applied onto the sample application pad and a labeled first molecule (detection agent, e.g., antibody) that binds to the analyte that is prefixed on the conjugate pad gets hydrated and starts flowing with the moving liquid. The test line contains, as the second molecule, pre-immobilized analyte (the same analyte as the analyte to be detected) that can bind specifically to labeled first molecule. The control line contains pre-immobilized third molecule (e.g., antibody) that can bind to the labeled first molecule. When the liquid sample reaches the test line, pre-immobilized analyte (second molecule) will bind to the labeled first molecule. Such binding principally happens when target analyte is absent from the sample or present in such a low quantity that some sites of labeled first molecule remain unbound by analyte. Analyte in the sample solution and analyte immobilized at the test line of strip compete to bind with labeled first molecule. Labeled first molecule that is not bound at the test line will be captured at the control line by the third molecule.

In the second layout of the competitive format, labeled analyte (as the labeled first molecule/detection agent) is dispensed at the conjugate pad while a second molecule (e.g., antibody) that can bind to the analyte is dispensed at the test line. After application of the sample solution a competition takes place between analyte in the sample and the labeled analyte to bind with the first molecule at the test line. The control line contains pre-immobilized third molecule (e.g., antibody) that can bind to the labeled analyte (first molecule).

There is also a hybrid format that modifies the direct format into a competitive form. In this hybrid, the strip is set up essentially as is the strip in a typical direct format LFA. To this is added a third line (analyte line) on the membrane between the test line and control line. The analyte line has analyte bound, generally via an immobilized molecule (e.g., antibody) that binds the analyte. This format involves a competition between analyte in solution and analyte pre-dispensed on the analyte line. In the case of very low concentration of the analyte in the sample, most of the labeled first molecules will remain unreacted and migrate to the analyte line, analyte present at analyte line will then capture these labeled first molecules. This will result in an intense color at analyte line and the rest of labeled first molecule will move to the control line and will produce a relatively less intense color. In the case of very high concentrations, most of the analyte will be captured at the test line and will be sandwiched in between labeled first molecule and the prefixed second molecule at the test line. This complex will then move and be captured by the third at the control line. In this case very few labeled first molecules will be retained at the analyte line. The less intense the color at analyte line, the higher the concentration of analyte present from the sample.

A multiplex detection format can be used for detection of more than one target species. The assay can be performed over a strip containing a number of test lines equal to the number of target species to be analyzed. It is highly desirable to analyze multiple analytes simultaneously under same set of conditions. The multiplex detection format is very useful in clinical diagnosis where multiple analytes which are inter-dependent in deciding about the stage of a disease are to be detected. Lateral flow strips for this purpose can be built in various ways, i.e., by increasing length and test lines on a conventional strip or by making other structures like stars or T-shapes. The shape of the strip for LFA can be chosen based on the number of target analytes. Miniaturized versions of LFA based on microarrays for multiplex detection of DNA sequences have several advantages such as less consumption of test reagents, requirement of lesser sample volume and better sensitivity.

2. Enzyme-Linked Immunosorbent Assay (ELISA)

Enzyme-Linked Immunosorbent Assay (ELISA) is also a common immunoassay, in which antibodies, peptides, proteins, and small molecules can be detected and quantified using a multi-well plate. Generally, enzyme-linked immunosorbent assay (ELISA) can also be used to detect one or more QS molecules in the sample. Antibodies will be specific for QS signals/molecules. For example, the bacterium Pseudomonas aeruginosa produces two QS signals: C4 Homoserine lactone and C12 homoserine lactone.

In some forms, antibodies specific for QS signals are utilized to identify these target molecules in the sample (e.g., sputum) with the popular diagnostic technique of ELISA. In some forms, antibodies are specific for QS signals from bacterium Pseudomonas aeruginosa including C4 Homoserine lactone and C12 homoserine lactone.

Preferably, competitive or inhibition ELISA assays are used to detect the specific QS signals. In some forms, the presence of one or more target QS molecules is determined by specific color change in the ELISA assays. Positive and negative controls are generally used to ensure the assays are working properly, for example, 2 colored bands indicate that the patient is negative for these QS signals whereas samples with one band indicate the presence of these QS signals. In the competition ELISA assays, the lack of band or color means a positive result for the presence of the target QS signals since the target QS molecules are competing for the binding with in the ELISA antibody; two bands means the sample is negative of the target QS molecules.

In some forms, reactivity of QS molecules to the antibody is compared using a test tube divided by different antibodies that will identify the type of QS molecules and therefore identify the causative bacterial involved in the infection.

3. Materials and Processing for Immunoassays

The sample can be, for example, sputum, tracheal, bronchoalveolar, and bronchial washes or lavage, blood, urine, and swabs. Generally, the samples can be extracted via liquid-liquid separation. For example, samples can be extracted by mixing the sample with dichloromethane (DCM) or a mixture of DCM and methanol. The ratio of sample to solvent can be, for example 4:1, 3:1, 2:1, 1:1, 1:2, 1:3, or 1:4. Preferably, the ratio of sample to solvent is 1:1. In the extraction the aqueous phase is removed and the organic phase (extracted sample) retained and further processed. In subsequent extractions of the organic phase, the extracted sample can be extracted with additional solvent. The ratio of extracted sample to solvent can be, for example 4:1, 3:1, 2:1, 1:1, 1:2, 1:3, or 1:4. Preferably, the ratio of extracted sample to solvent is 1:1. A total of two, three, for, five, six, or seven solvent extractions can be performed. Preferable, four solvent extractions are performed. At different stages, the sample, extraction mix, or extracted sample can be centrifuged to separate layers and/or separate particulates. The final extracted sample can be dried (to remove the solvent), filtered, or filtered and dried. For example, the extracted sample can be filtered through, for example, a 0.2 μm filter and then dried at, for example room temperature. The dried extracted sample can then be resuspended in, for example, acetyl nitrile.

The antibodies specific for a quorum sensing molecule can be, for example, mouse monoclonal antibodies. For detecting and assessing P. aeruginosa quorum sensing molecules (N-acyl homoserine lactones) an anti N-acyl homoserine lactone antibody can be used. For example, for detection of the P. aeruginosa quorum sensing molecule 3-oxo-dodecanoyl-homoserine lactone (N-3-oxo-C12-HSL), the anti N-acyl homoserine lactone antibody clone RS2-1G9 can be used (Absolute antibody, Boston, Mass., USA). Alternatively, an antibody or antibody fragment having the CDRs of this antibody can be used. The clone RS2-1G9 antibody was raised in mice against immunogen 4-methoxyphenyl amide acyl homoserine lactone analog as the immunogen, and binds to 3-oxo-dodecanoyl-homoserine lactone (N-3-oxo-C12-HSL) (Kaufmann et al., J. American Chemical Society 128(9):2802-2803 (2006)). The RS2-1G9 antibody interferes with quorum sensing and was shown to protect murine bone marrow derived macrophages from the cytotoxic P. aeruginosa quorum sensing signaling molecule N-3-oxo-dodecanoyl-homoserine lactone.

The antibodies used in the immunoassays are generally diluted to an appropriate level, which is generally based on the concentration and affinity of the antibody. For example, the antibodies can be used at a dilution of 1:50, 1:100, 1:200, 1:300, 1:400, 1:500, 1:750, 1:1000, 1:2000, 1:3000, 1:4000, 1:5000, 1:6000, 1:7000, 1:8000, 1:9000, or 1:10,000. Preferred dilution for antibodies specific for quorum sensing molecules are 1:50, 1:100, and 1:200. Preferred dilutions for secondary antibodies are 1:7000, 1:8000, 1:9000, or 1:10,000.

For labeled antibodies, such as antibodies used in detection agents, the label can be added in a manner appropriate for the label. A useful way to label an antibody is to bind the antibody with a labeled antibody that binds to a generic feature of the first antibody. For example, where the first antibody is a mouse antibody, a labeled anti-mouse antibody can be used. For example, to label a mouse antibody, a labeled goat anti-mouse IgG1 can be used. For example, a goat anti-mouse IgG1 gold conjugated polyclonal antibody can be used. In the disclosed immunoassays, the labeled antibody can be the detection agent. For a LFA, this detection agent can be spotted on the conjugation pad.

To capture the detection agent during the assay, an antibody that binds to a generic feature of the antibody in the detection agent can be used. For example, where the antibody in the detection agent is a mouse antibody, an anti-mouse antibody can be used. For example, a goat anti-mouse IgG1 can be used. For example, a goat anti-mouse IgG1 monoclonal antibody can be used. This antibody would be spotted on the membrane of the strip, generally at the control line, but also or alternatively at the test line depending on the format. The spotted antibodies and other reagents generally are allowed to dry in place. Generally, the capture antibody is used at a standard concentration and/or amount. For example, the capture antibody can be used at a concentration of 8 mg/ml, 8.5 mg/ml, 9 mg/ml, 9.5 mg/ml, 10 mg/ml, 10.5 mg/ml, 11 mg/ml, 11.5 mg/ml, 12 mg/ml, 12.5 mg/ml, 13 mg/ml, 13.5 mg/ml, or 14 mg, ml. Preferably, the capture antibody is used at a concentration of 11.5 mg/ml.

In immunoassays where the quorum sensing molecule is spotted or immobilized on the strip, the quorum sensing molecule can be applied directly or a conjugate of the quorum sensing molecule can be applied. For example, for smaller quorum sensing molecules and for non-protein quorum sensing molecules, it can be useful to conjugate the quorum sensing molecule to a protein. For example, the quorum sensing molecule can be conjugated to bovine serum albumin (BSA). The protein aids in attachment and retention of the quorum sensing molecule. In the particular case of assays for detecting C12HSL, a C12HSL conjugate can be used (e.g., the C12HSL BSA conjugate from CellMosaic Woburn, Mass., USA). and used at full strength. This was applied to nitrocellulose paper (GE Health care Life Science) to detect the present or absent of quorum sensing (QS) molecules. The lack of a signal means that the target analyte is present in the patient sample and competition inhibition has occurred between the mouse monoclonal antibody anti-N-acyl homoserine lactones and the analyte.

B. Analytical Assays

1. Thin Layer Chromatography (TLC)

Thin layer chromatography (TLC) is a chromatographic technique used to separate the components of a mixture using a thin stationary phase supported by an inert backing. TLC is an analytical tool widely used because of its simplicity, relative low cost, high sensitivity, and speed of separation. TLC functions on the same principle as all chromatography: a compound will have different affinities for the mobile and stationary phases, and this affects the speed at which it migrates. The goal of TLC is to obtain well defined, well separated spots. This method can take a sample, such as a sputum sample, from a patient presenting with pneumonia-like symptoms as determined by a healthcare professional. The samples are generally minimally processed. In some forms, the sample processing involves centrifugation and filtration, for example, through a 0.2 micron filter. In other forms, samples may also undergo a process such as liquid-liquid separation to reduce total volume and concentrate the target molecules. Excess solvent is generally evaporated away.

TLC usually consists of a stationary phase and a mobile phase. The stationary phase is typically silica gel or C18 reverse phase plate that is applied to a flat surface such as plastic, glass, or paper. On to this flat surface there is a thin layer of the stationary phase material that will be used to separate out a mixture into its individual components based on polarity. In some forms, the test sample will be blotted to the bottom of the TLC plate at least one centimeter from the bottom of the plate. Known control samples containing one or more of the target QS molecules are generally applied alongside as positive controls. The target QS molecules include but not limited to Pseudomonas aeruginosa QS molecules such as C4 HSL and C12 HSL and Staphylococcus aureus QS molecules such as AIP-1 and AIP-2.

In one example, the prepared TLC plate is placed vertically into a glass jar into which a mobile solvent system will be added. This mobile phase contains one or more volatile solvents including but not limited to acetone, methanol, dichloromethane, chloroform, petroleum ether and hexane. The volume added to the glass jar should generally be below the spotted test samples. The jar will then be covered in order to create a closed system. The samples will then travel up the TLC plate via capillary action. After all the test samples components have been separated on the plate. The plate will be removed to air dry for about 2 minutes. The unknown samples will be compared to the known control samples. They will be matched based on reference factor values. Here, the distance traveled of the spot divided by the overall distance of the plate will be used to calculate. The location traveled will be made visible using a UV light or a stain. The matching of the known control against the unknown sample will identify the sample as having the one or more target QS molecules, which in turn indicates the causative agent of the bacterial infection. For example, samples identified as containing one or more of N-acyl homoserine lactone such as C4 Homoserine lactone and C12 homoserine lactone indicate the patient from which the sample is collected from is positive for Pseudomonas aeruginosa infection.

2. High Performance Chromatography Method (HPLC)

High Performance Liquid Chromatography (HPLC) is a form of column chromatography that pumps a sample mixture or analyte in a solvent (known as the mobile phase) at high pressure through a column with chromatographic packing material (stationary phase).

High-Performance Liquid chromatography can be used to identify the bacterial QS molecule(s) in the sample. The samples will be injected into the HPLC containing a silica gel or reverse phase column in order to identify the sample by comparing the peaks on the screen. The mobile phase can be acetonitrile and methanol at an increased concentration gradient over the lifespan of the experimental cycle. Reference samples are also run before the patients sample to determine where in the gradient the molecules of interest migrate. HPLC is a specialized sensitive chromatography method for detecting one or more QS molecules.

C. Solid Supports

Solid supports are used to hold or immobilize the disclosed proteins, peptides, analytes, antigens, antibodies, and other components. Solid supports are solid-state substrates or supports with which molecules (such as peptides and proteins) or other components used in, or produced by, the disclosed methods can be associated. Molecules can be associated with solid supports directly or indirectly. For example, peptides can be bound to the surface of a solid support. An array is a solid support to which multiple peptides or other molecules have been associated in an array, grid, or other organized pattern.

Solid-state substrates for use in solid supports can include any solid material with which components can be associated, directly or indirectly. This includes materials such as acrylamide, agarose, carboxylated poly(vinyl chloride) (CPVC), cellulose acetate membrane, cellulose nitrate (CN) membrane, cellulose, collagen, filter paper (Whatman), fluorocarbons, functionalized silane, Glass fiber filters (GFC) (A,B,C), glass, glycosaminoglycans, gold, latex, mixed cellulose ester membrane, nitrocellulose, nylon, plastic, polyamino acids, polyanhydrides, polycarbonates, polyethersulfone (PES) membrane, polyethylene oxide, polyethylene vinyl acetate, polyethylene, polyethylimine coated GFCs, polyglycolic acid, polylactic acid, polymethacrylate, polyorthoesters, polypropylene, polypropylfumerate, polysilicates, polystyrene, polyvinylidene fluoride (PVDF), porous mylar or other transparent porous films, PTFE membrane, silicon rubber, teflon, andultrafiltration membranes of poly(vinyl chloride) (PVC). Solid-state substrates can have any useful form including beads, bottles, chemically-modified glass slides, column matrix, cross-linked polymer beads, dishes, fibers, mass spectrometer plates, membranes, microparticles, microtiter dishes, particles, shaped polymers, slides, sticks, test strips, thin films, thin membranes, and woven fibers, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

An array can include a plurality of molecules, compounds or peptides immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification.

Although useful, it is not required that the solid support be a single unit or structure. A set of molecules, compounds and/or peptides can be distributed over any number of solid supports. For example, at one extreme, each component can be immobilized in a separate reaction tube or container, or on separate beads or microparticles.

Methods for immobilization of proteins and peptides to solid-state substrates are well established.

Each of the components immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

D. Detection Agents

A detection agent is a specific binding molecule that also comprises or is coupled to a detection element. The specific binding molecule can be referred to as the affinity portion of the detection agent and the detection element is referred to as the detection element portion of the detection agent. As used herein, a specific binding molecule is a molecule that interacts specifically with a particular molecule or moiety. The molecule or moiety that interacts specifically with a specific binding molecule is referred to herein as a target molecule. Quorum sensing molecules are examples of target molecules. It is to be understood that the term target molecule refers to both separate molecules and to portions of molecules, such as an epitope of a protein that interacts specifically with a specific binding molecule. For example, the IgG or IgM determinant of an antibody can be the portion of an antibody that a specific binding molecule interacts with. Antigens, antibodies, either member of a receptor/ligand pair, and other molecules with specific binding affinities are examples of specific binding molecules, useful as the affinity portion of a detection agent. A detection agent with an affinity portion that is an antibody can be referred to herein as a detection antibody. By coupling a detection element to such specific binding molecules, binding of a specific binding molecule to its specific target can be detected by detecting the detection element. A detection agent that interacts specifically with a particular target molecule is said to be specific for that target molecule. For example, a detection agent with an affinity portion which is an antibody that binds to a particular antigen is said to be specific for that antigen. The antigen is the target molecule. Detection agents are also referred to herein as detection molecules.

A preferred form of detection agent is an antibody specific for the analyte to be detected. Another preferred form of detection agent is the analyte to be detected.

E. Detection Elements

To aid in detection of analytes such as quorum sensing molecules, detection elements can be directly can be associated with or coupled to detection agents. As used herein, a detection element is any molecule that can be associated with a detection agent, directly or indirectly, and which results in a measurable, detectable signal, either directly or indirectly. Many such labels for are known to those of skill in the art. Examples of suitable detection elements include radioactive isotopes, fluorescent molecules, phosphorescent molecules, enzymes, antibodies, and ligands.

The disclosed detection elements can be part of, and detectable with, enzyme-linked detection systems. Enzyme-linked detection generally involves an enzyme as a label or tag on a component where the presence of the enzyme (and thus of the analyte with which the enzyme is associated) is detected by having the enzyme convert an enzymatic substrate into a form that produces a detectable signal. For example, analytes labeled or associated with alkaline phosphatase can be detected by adding the chemiluminescent substrate CSPD (Tropix, Inc.). The fluorescent reaction product can then be detected. Preferred forms of detection elements are enzymes, such as alkaline phosphatases and peroxidases, for use in an enzyme-linked detection system.

Examples of suitable fluorescent labels include fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels for combinatorial multicolor coding are FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, Oreg. and Research Organics, Cleveland, Ohio.

Detection elements such as biotin can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo [3.3.1.1^{3,7}]decane]-4-yl) phenyl phosphate; Tropix, Inc.).

Molecules that combine two or more of these detection elements are also considered detection elements. Any of the known detection elements can be used with the disclosed detection agents. Methods for detecting and measuring signals generated by detection elements are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection element coupled to the antibody. Such methods can be used directly in the disclosed method of amplification and detection. As used herein, detection agents are molecules which interact with amplified nucleic acid and to which one or more detection elements are coupled.

F. Reporter Agents

Reporter agents are molecules, compounds, or components that can facilitate detection of detection elements. Reporter agents are most useful when the detection element does not produce a detectable signal or a conveniently detectable signal. In some forms, the reporter agent can generate or be converted into a detectable signal or as molecule, compound, or component that produces a detectable signal. For example, if the detection element is an enzyme, the reporter agent can be a substrate for the enzyme here the enzymatic product of the reporter agent is or produces a detectable signal. In some forms, the reporter agent can be or comprise a detectable signal. In these forms, association of the reporter agent with the detection element associates the detectable signal with the detection agent. This essentially labels the detection agent with the detectable signal of the reported agent.

Preferred reporter agents are enzymatic substrates, such as substrates that produce a detectable signal upon reaction with their respective enzyme. Such reporter agents are thus part of an enzyme-linked detection system, with the enzyme associated with or coupled to a detection agent (with the enzyme thus serving as a detection element).

### III. Methods

Bacteria and fungi have been shown to produce quorum sensing signals as a way of orchestrating joint responses. They produce these indicators as they are colonizing the patients and it is the trigger surrounding bacteria and fungi that they will start the attack. Additionally, the bacteria and fungi will produce the QS when these are just beginning to congregate together in a confined location. These organisms produce external chemical signals in a particular location. When the concentration of these signs reaches a minimum concentration, the signal will diffuse back into the cell and triggers group mediated gene expression. These genes trigger the production of a whole myriads of reactions such as toxin production, pathogenicity, virulence, biofilm development, antibiotic production, efflux pump express, rhamnolipid production. In some bacteria it activates bioluminescence, pigment production, swimming and swarming. In pathogenic bacteria will start the expression of pathogenic genes. These genes include: biofilm development, rhamnolipid production, toxin production, pigments, antibiotic production to name a few. The pool of these signals can be found in sputum, urine, blood, stool, saliva, ear rinses, among others.

Disclosed are methods for determining a bacterial infection caused by particular bacteria, such as Pseudomonas aeruginosa and related species, in a subject. In some forms, the subject is having one or more symptoms associated with pneumonia, bronchitis, and/or UTI. The disclosed methods are also suitable for detecting the presence of bacterial colonization, particular that of P. aeruginosa, and/or confirming an active infection by P. aeruginosa.

The particular bacteria detected or assessed in the disclosed methods, systems, devices, and kits can be any bacterial species, group of species, genus, group of genera, family, group of families, order, group of orders, class, group of classes, phyla, or gram-staining division. Generally, the QS molecule(s) assessed will be characteristic for the type or set of bacteria to be detected or assessed. In some cases, particular QS molecules or combinations of QS molecules will be characteristic of the type or set of bacteria. Given the context and source of the sample, the QS molecules need only be characteristic of the bacteria in the context and source of the sample.

In some forms, the microbe can be Pseudomonas aeruginosa, Klebsiella pneumoniae, Clostridium difficle, Streptococcus pneumoniae, Staphylococcus aureus, Cryptococcus neoformans, Candida albicans, Haemophilus influenza, Mycobacterium tuberculosis, Legionella pneumophila, or combinations thereof. In some forms, the bacteria can be Pseudomonas aeruginosa, Klebsiella pneumoniae, Clostridium difficle, Streptococcus pneumoniae, Staphylococcus aureus, Haemophilus influenza, Mycobacterium tuberculosis, Legionella pneumophila, or combinations thereof. In some forms, the fungi can be Cryptococcus neoformans, Candida albicans. In some cases, the organism to be detected is microbial fungi that produces QS molecules. In such cases and only for descriptive purposes, references herein to bacteria to be detected should be considered references to such microbial fungi. All such references to bacteria optionally can be explicitly limited to bacteria (such as by explicitly excluding microbial fungi).

Disclosed are methods that provide fast, reliable, accurate diagnosis of bacterial infection caused by particular bacterial, such as Pseudomonas aeruginosa. In some forms, the methods include a colorimetric bioassay for fast read-out. The visual read-out format and simplicity make the claimed methods well suited for rapid and accurate detection of causative agents in remote areas, clinics, and fields with minimal access to laboratory facilities, and greatly benefit the point-of-care applications, especially during infectious disease outbreaks.

Disclosed are a variety of methods each of which can include assaying samples, such as sputum, urine, or other fluid, from a subject, which can in turn produce an assay output, which can be used. The methods can also involve transmitting the assay output to a recipient. Typically the assays can be an in vitro or ex vivo assay. Any type of assays suitable for detecting and/or measuring the amounts of molecules, such as Lateral Flow, Thin Layer Chromatography Method (TLC), High Performance Chromatography Method (HPLC), Enzyme-linked immunosorbent assay (ELISA) can be used. The methods can involve contacting various reagents together, as well as using controls, such as positive controls, and they can involve normalizing as well as standards. In any form disclosed, it is understood that other steps or forms can optionally be included or removed. In some forms, the methods can utilize bodily fluids and/or cells and can involve steps of comparing different results or molecules or materials or substances, or any disclosed aspect herein, by for example comparing whether they are higher, or inhibited, lower, reduced, or prevented, for example. The methods can also include the step of obtaining results or samples or the like. The methods can also include the step of determining and diagnosing, as well as looking at the confidence of a particular result or conclusion to determine its accuracy.

The methods typically revolve around bacterial infections, such as bacterial pneumonias, bronchitis and urinary tract infection. The methods can also include prescribing treatments, such as a prescription, such as those provided by a physician.

The methods can also include treatments and treatment options, of for example antibiotics alone or in co-application with other molecules such as pharmaceuticals or pro-drugs, having pharmacological activity. Treatments can also seek to provide a therapeutically effective amount of a drug.

The methods can typically detect the presence of one or more QS molecules associated with particular bacteria, such as P. aeruginosa. In some forms, the QS molecule is an N-acyl homoserine lactone (AHL) such as N-butyryl homoserine lactone (C4-HSL), N-hexanoyl homoserine lactone (C6-HSL), N-(3-oxo)-hexanoyl homoserine lactone (3-oxo-C6-HSL), N-octanoyl homoserine lactone (C8-HSL), N-(3-oxo)-octanoyl homoserine lactone (3-oxo-C8-HSL), N-decanoyl homoserine lactone (C10-HSL), N-dodecanoyl homoserine lactone (C12-HSL), N-(3-oxo)-dodecanoyl homoserine lactone (3-oxo-C12-HSL), N-tetradecanoyl homoserine lactone (C14-HSL), and combinations thereof. In some forms, the QS molecule is N-butanoyl-L homoserine lactone (C4-HSL) and/or N-(3-oxododecanoyl)-L-homoserine lactone (3-oxo-C12-HSL).

A. Methods of Determining the Causative Agent of an Infection

The disclosed methods are suitable for use in identifying the causative agent of an infection, for example, in subjects with bacterial pneumonias, bronchitis and urinary tract infection. Patients may have acquired pneumonia via many different routes such as hospital acquired pneumonia (HAP), ventilator acquired pneumonia (VAP), and community acquired pneumonia (CAP). In some forms, the methods can identify causative bacteria including P. aeruginosa by detecting the presence of one or more QS molecules specifically associated with P. aeruginosa. In some forms, the QS molecule is C4 homoserine lactone and/or C12 homoserine lactone, e.g., N-butanoyl-L homoserine lactone (C4-HSL) and/or N-(3-oxododecanoyl)-L-homoserine lactone (3-oxo-C12-HSL).

The methods can assist physician in confirming an active infection, in particular those caused by Pseunomonas aeruginosa.

B. Methods of Diagnosing and/or Treatment

Disclosed are methods of diagnosing a bacterial infection particular those caused by Pseunomonas aeruginosa in a subject. The methods include measuring the levels of at least one biomarker in a sample from the subject, wherein the presence and/or increased levels of each of the measured biomarkers relative to a control without P. aeruginosa infection means the bacterial infection in the subject is caused by P. aeruginosa, producing a diagnosis result. In some forms, the biomarker is C4 homoserine lactone and/or C12 homoserine lactone.

Disclosed are methods of diagnosing a P. aeruginosa infection by detecting the presence of one or more QS molecules associated with P. aeruginosa. In some forms, the QS molecule is C4 homoserine lactone and/or C12 homoserine lactone, e.g., N-butanoyl-L homoserine lactone (C4-HSL) and/or N-(3-oxododecanoyl)-L-homoserine lactone (3-oxo-C12-HSL).

The methods can further include obtaining the diagnosis result and prescribing an antibiotic for the subject if the presence and/or increased levels of C4 homoserine lactone and/or C12 homoserine lactone is detected. In some forms, the methods comprise obtaining the prescription, and using the antibiotic as it was prescribed. In some forms, the methods comprise obtaining the prescription and collecting the antibiotic of the prescription, placing it in a canister, and selling the antibiotic in the canister, such as filling the prescription.

The method can further include guiding treatment regimens as to whether to initiate, continue, or discontinue treatment of a bacteria-related condition of interest in a subject is provided. In some forms, the bacteria-related condition of interest is a bacterial infection caused by P. aeruginosa.

In some cases, P. aeruginosa is detected in the sample collected from a subject, then the subject is prescribed and/or administered one or more antibiotics for treating bacterial infection caused by P. aeruginosa. Once the P. aeruginosa infection is established by the disclosed methods, physicians can treat the infection accordingly, for example, as reviewed in Matteo Bassetti et al., (Matteo Bassetti et al., Drugs Context. 2018; 7: 212527). Eight categories of antibiotics are commonly used to treat P. aeruginosa infections including aminoglycosides (gentamicin, tobramycin, amikacin, netilmicin), carbapenems (imipenem, meropenem), cephalosporins (ceftazidime, cefepime), fluoroquinolones (ciprofloxacin, levofloxacin), penicillin with β-lactamase inhibitors (BLI) (ticarcillin and piperacillin in combination with clavulanic acid or tazobactam), monobactams (aztreonam), fosfomycin and polymyxins (colistin, polymyxin B).

In other cases, P. aeruginosa is not detected in the sample collected from a subject, i.e., same as the negative control, then the subject is refrained from prescribing or administering an antibiotic for treating P. aeruginosa.

C. Methods of Monitoring

The disclosed methods include methods of monitoring a subject having Pseudomonas aeruginosa infection. The methods typically comprises the step of treating the subject for P. aeruginosa infection and followed by performing any of the disclosed methods.

### IV. Kits

Kits for diagnosis of bacterial infection and/or monitoring the status of bacterial infection using detection molecules that specifically bind a quorum sensing target molecule or a quorum sensing-associated target molecule are described. In particular, kits for detecting one or more quorum sensing target molecules produced by P. aeruginosa are provided. In some forms, the detection molecules, e.g., antibodies, bind specifically to one or more molecules involved in quorum sensing including N-acyl homoserine lactone (AHL). In some forms, the QS molecule is an N-acyl homoserine lactone (AHL) such as N-butyryl homoserine lactone (C4-HSL), N-hexanoyl homoserine lactone (C6-HSL), N-(3-oxo)-hexanoyl homoserine lactone (3-oxo-C6-HSL), N-octanoyl homoserine lactone (C8-HSL), N-(3-oxo)-octanoyl homoserine lactone (3-oxo-C8-HSL), N-decanoyl homoserine lactone (C10-HSL), N-dodecanoyl homoserine lactone (C12-HSL), N-(3-oxo)-dodecanoyl homoserine lactone (3-oxo-C12-HSL), N-tetradecanoyl homoserine lactone (C14-HSL), and combinations thereof. In some forms, the QS molecule is N-butanoyl-L homoserine lactone (C4-HSL) and/or N-(3-oxododecanoyl)-L-homoserine lactone (3-oxo-C12-HSL).

The kits contain some or all of the materials needed to measure each of C4 Homoserine lactone and C12 homoserine lactone, alone, in series, or simultaneously.

In some forms, the kit contains a test strip that gives a positive reading only when the one or more target QS signals are detected. Readout of the test strip would allow the clinician to have a sensitivity and specificity to determine whether P. aeruginosa is present or not, and whether the patient has recovered in convalescence or not.

The kits can give one single positive reading if both biomarkers are positive or the kits can give individual positive readings for each of the two biomarkers separately. In some forms, the kit can have a combination of the aforementioned. The test strip can have a spot for a positive reading for both biomarkers together and two individual spots for positive readings of each biomarker separately.

In some forms, the kit includes one or more lateral flow device for detecting the presence of one or more QS molecules, particularly those associated with P. aeruginosa.

The kits generally contain instructions on how to use each component of the kits as well as how to interpret the results.

The disclosed compositions and methods can be further understood through the following numbered paragraphs.

1. A kit for detecting a quorum sensing molecule comprising:

a solid support, wherein a first antibody is immobilized on the solid support; and

a detection agent, wherein the detection agent comprises a detection element,

wherein either (a) the detection agent further comprises a second antibody specific for the quorum sensing molecule and the first antibody is specific for the second antibody or (b) the detection agent further comprises the quorum sensing molecule and the first antibody is specific for the quorum sensing molecule.

2. The kit of paragraph 1, wherein the solid support is in the form of a test strip.

3. The kit of paragraph 2, wherein the test strip is an immunochromatographic test strip.

4. The kit of paragraph 2 or 3, wherein the test strip comprises a conjugate pad and a membrane, wherein the membrane comprises a test line and a control line, wherein the test line is closer to the conjugate pad than the control line.

5. The kit of paragraph 4, wherein the detection agent comprises the detection element and the second antibody, wherein the first antibody is specific for the second antibody, wherein the first antibody is immobilized on the membrane at the control line, wherein the quorum sensing molecule is immobilized on the membrane at the test line, wherein the detection agent is detachably fixed on the conjugate pad.

6. The kit of paragraph 4, wherein the detection agent comprises the detection element and the quorum sensing molecule, wherein the first antibody is specific for the quorum sensing molecule, wherein the first antibody is immobilized on the membrane at both the control line and the test line, wherein the detection agent is detachably fixed on the conjugate pad.

7. The kit of paragraph 4, wherein the detection agent comprises the detection element and the second antibody, wherein the first antibody is specific for the second antibody, wherein the first antibody is immobilized on the membrane at the control line, wherein a third antibody specific for the quorum sensing molecule is immobilized on the membrane at the test line, wherein the second antibody and the third antibody bind to the quorum sensing molecule noncompetitively, wherein the detection agent is detachably fixed on the conjugate pad.

8. The kit of paragraph 7, wherein the membrane further comprises an analyte line, wherein the analyte line is disposed between the test line and the control line, wherein the quorum sensing molecule is immobilized on the membrane at the analyte line.

9. The kit of any one of paragraphs 1-8, wherein the kit further comprises a reporter agent, wherein the reporter agent can facilitate detection of the detection element.

10. The kit of paragraph 9, wherein the second antibody, the reporter agent, and the detection element are components of an enzyme-linked immunosorbent assay (ELISA) system.

11. The kit of any one of paragraphs 1-10, wherein the detection element is an enzyme, wherein the enzyme catalyzes a reaction that can produce a detectable signal.

12. The kit of paragraph 11, wherein the reporter agent is an enzymatic substrate for the enzyme, wherein the enzyme can act on the reporter agent to produce the detectable signal.

13. A method of detecting the existence of a significant bacterial infection in a subject, the method comprising:

bringing into contact a sample from the subject and the solid support of the kit of any one of paragraphs 1-12; and

detecting the detection element on the solid support,

wherein the presence or absence of the detection element on the solid support or at a particular location on the solid support indicates the presence of the quorum sensing molecule in the sample, wherein the indication of the presence of the quorum sensing molecule in the sample indicates the existence of a significant bacterial infection in the subject.

14. The method of paragraph 13, wherein the solid support is in the form of a test strip.

15. The method of paragraph 14, wherein the test strip is an immunochromatographic test strip.

16. The method of paragraph 14 or 15, wherein the test strip comprises a conjugate pad and a membrane, wherein the membrane comprises a test line and a control line, wherein the test line is closer to the conjugate pad than the control line.

17. The method of paragraph 16, wherein the detection agent comprises the detection element and the second antibody, wherein the first antibody is specific for the second antibody, wherein the first antibody is immobilized on the membrane at the control line, wherein the quorum sensing molecule is immobilized on the membrane at the test line, wherein the detection agent is detachably fixed on the conjugate pad.

18. The method of paragraph 17, wherein the absence of the detection element at test line on the solid support indicates the presence of the quorum sensing molecule in the sample.

19. The method of paragraph 16, wherein the detection agent comprises the detection element and the quorum sensing molecule, wherein the first antibody is specific for the quorum sensing molecule, wherein the first antibody is immobilized on the membrane at both the control line and the test line, wherein the detection agent is detachably fixed on the conjugate pad.

20. The method of paragraph 19, wherein the absence of the detection element at test line on the solid support indicates the presence of the quorum sensing molecule in the sample.

21. The method of paragraph 16, wherein the detection agent comprises the detection element and the second antibody, wherein the first antibody is specific for the second antibody, wherein the first antibody is immobilized on the membrane at the control line, wherein a third antibody specific for the quorum sensing molecule is immobilized on the membrane at the test line, wherein the second antibody and the third antibody bind to the quorum sensing molecule noncompetitively, wherein the detection agent is detachably fixed on the conjugate pad.

22. The method of paragraph 21, wherein the membrane further comprises an analyte line, wherein the analyte line is disposed between the test line and the control line, wherein the quorum sensing molecule is immobilized on the membrane at the analyte line.

23. The method of paragraph 21 or 22, wherein the presence of the detection element at test line on the solid support indicates the presence of the quorum sensing molecule in the sample.

24. A diagnostic tool for elucidation of infectious diseases by identification of bacteria and fungi.

25. The diagnostic tool of paragraph 24, using a characterized detection molecule and at least one detecting field.

26. Target molecules for the diagnostic tool of paragraph in 24 which are C12-BSA, C12 HCL, C4 HCL, and associated antibodies of these group, optionally including, but not limited to, items presented in Table 1.

27. The diagnostic tool of paragraph 24, for gram positive quorum sensing and for gram negative quorum sensing.

28. A method of using the diagnostic tool of paragraph 24 using sputum, blood, urine, swabs, and other respiratory samples, such as tracheal, bronchoalveolar, and bronchial washes or lavage.

29. The method of paragraph 28 using Lateral Flow, TLC, HPLC, or ELISA.

30. The method of paragraph 29 using Lateral flow.

31. The method of paragraph 29 using TLC.

32. The method of paragraph 29 using HPLC.

33. The method of paragraph 29 using ELISA.

34. The diagnostic tool of paragraph 24, wherein the infectious diseases are defined as causative of specific transmittable illness that are originated by either bacteria or fungi.

35. The diagnostic tool of paragraph 34, wherein the infectious diseases include, but are not limited to, respiratory ailments such as pneumonia, UTI, bronchitis, wounds, abscess, and others.

36. The diagnostic tool of paragraph 34 used to define bacteria and fungi, optionally defining gram morphology, gram stain for bacterial and fungus genus and or species.

37. The diagnostic tool of paragraph 36 used to further characterize bacteria and fungi by detecting and/or distinguishing colonialization vs infection, quantifying vs qualifying, multi microbe infections, colony or poly-colonial or multiple species infection (how many types of bacteria reside in the infection).

38. The diagnostic tool of paragraph 24 using quorum sensing molecules as a detecting moiety.

39. The diagnostic tool of paragraph 38 for gram positive bacteria, using oligo peptides for detection.

40. The diagnostic tool of paragraph 38 for gram negative bacteria existence, using quorum sensing molecule N-Acyl homoserine lactone for identification.

41. The diagnostic tool of paragraph 37, in relation to fungi infection, using quorum sensing auto inducers for detection.

42. A method of developing of diagnostic tools for bacterial species identification.

43. A rapid method for detection of virulent bacterial species using quorum sensing molecules.

44. A rapid method using the diagnostic tool of paragraph 27 and using samples from sputum, urine, or other body fluids.

45. A rapid method to measure inhibition signal.

46. A rapid method to qualify signal.

47. A rapid method to quantify quorum signal either using antibodies or the signal directly.

48. A rapid method to characterize the bacterial genus or species.

49. A rapid method for diagnosis of pneumonia, UTI, wound infections, among others.

### EXAMPLES

### Example 1: Detection of Quorum Sensing Molecules in Patient Samples Via Dot Blot

### Material and Methods

Reagents

Primary antibody that recognizes the 297-Dalton compound of interest was used at a 1:8000 and 1:9000 dilution. This is produced by P. aeruginosa bacteria and is excreted out of the cell during the infection cycle. A gold conjugated goat anti-mouse antibody was used as a secondary antibody without dilution. We have also used an HRP antibody at 1:100 concentration, which allowed us to detect the primary antibody at a 1:9000 dilution. FF-120 Whatman filter paper (GE) was used for dot blotting.

Sample Extraction

A 1 ml of patient sputum sample was extracted via liquid-liquid separation with dichloromethane and methanol. The dichloromethane layer was collected, dried in a fume hood and then re-suspended into 1 ml either 60/40 Acetonitrile/water (Sample No. 1-15) or PBS (Sample No. 16-19).

Patient Samples

Patient samples were graciously acquired through a collaboration with a local hospital. These were samples taken from Cystic fibrosis patients positive with Pneumonia. An array of samples were collected as either tracheal, bronchoalveolar lavage, bronchial washes, or sputum samples described in Table 1. These samples were used to perform culture identification tests. These were completed by the hospital and the results were recorded. 25 patient samples were tested: 19 positive samples, and 6 negative samples. The remainder of the samples were used for further experimentation. Table 1 describes the specific details of the patient samples. Extensive details about age, gender, and other ailments were not included in this first set of samples.

Dot Blot Methodology

Briefly, a competitive Dot blot assay was completed on FF-120 Whatman filter paper. BSA-Conjugate, BSA blocking agent, off the shelf primary antibody used at 1:8000 and 1:9000 concentrations. A gold-conjugated antibody was used at the 1:8000 dilution. HRP goat anti-mouse antibody was used to detect primary antibody at a 1:9000 dilution.

### Results

Positive control was tested with primary antibody at a 1:1000 dilution and 1:9000 dilution. Samples 1-19 are P. aeruginosa positive patient samples; and samples N-1 to N-6 are six negative patients.

On the competitive dot blot, a lack of color indicates that the compound of interest is in the sample, while a colored spot indicates that there is no compound present in the sample. Samples 1-15, with the exception of sample 9, showed that the antigen of interest blocked the binding of the primary antibody to the BSA-conjugate. Sample 9 showed color primarily due to the concentration of the primary antibody being too high. Samples 16-19 required further optimization.

The dot blot data showed the immunoassays are viable point-of-care detection methods for providing fast and accurate results for detecting the presence of N-acyl homoserine lactone, and thus identifying patients with P. aeruginosa infection.

TABLE 1
Patient IDs and types of samples collected from each patient.

| | | Type of sample collected from |
|---|---|---|
| Sample No. | Sample ID | patient |
| 1 | PA-1 | Sputum |
| 2 | PA-2 | Bronchial Washing |
| 3 | PA-3 | Bloody Sample |
| 4 | PA-4 | Sputum |
| 5 | PA-5 | Bronchoalveolar lavage |
| 6 | PA-6 | Tracheal |
| 7 | PA-7 | Tracheal |
| 8 | PA-8 | Unsure-appears to be Tracheal |
| 9 | PA-9 | Tracheal |
| 10 | PA-10 | Sputum |
| 11 | PA-11 | Tracheal |
| 12 | PA-12 | Tracheal |
| | P.aeruginosa, | |
| | K.pneutnoniae | |
| 13 | PA-13 | Sputum Expectorated |
| 14 | PA-14 | Tracheal |
| 15 | PA-15 | Bronchial Washing |
| 16 | PA-16 | Sputum |
| 17 | PA-17 | Sputum |
| 18 | PA-18 | Sputum |
| 19 | PA-19 | Sputum |
| N-1 | Gram positive | Tracheal |
| | cocci- S.aureus | |
| N-2 | Gram negative- | Tracheal |
| | K.pneutnononiae | |
| N-3 | No Bacterial | Tracheal |
| | growth seen | |
| N-4 | No Bacterial | Tracheal |
| | growth seen | |
| N-5 | No Bacterial | Tracheal |
| | growth seen | |
| N-6 | Gram negative- | Tracheal |
| | K.pneutnoniae | |

### Example 2: Detection of Quorum Sensing Molecules in Patient Samples Via Lateral Flow

### Material and Methods

Human Sample

The human sample were graciously provided by a local hospital. These Cystic Fibrosis (CF) patients came to the Infectious disease specialist showing clinical manifestation of pneumonia. Sputum, tracheal wash and bronchial lavage were collected from the patients. The analytes were extracted from the samples and stored at 4 degrees. Briefly, samples were extracted using equal parts of (a) Dichloromethane (DCM) (Fisher Scientific, USA) and (b) the patient sample (Fisher Scientific, USA). The aqueous layer was removed after mixing for few seconds and spinning. The organic phase was mixed with equal volume of DCM. This procedure was repeated three times. Then the organic phase was centrifuged at 4,000 RPM for three minutes, purified thru a 0.2 μm filter (Fisher Scientific, USA), evaporated at room temperature, resuspended in Acetyl Nitrile (Fisher Scientific, USA) and stored at 4 degrees until the test was performed.

Antibodies

The mouse monoclonal antibody anti N-acyl homoserine lactones (Absolute antibody, Boston, Mass., USA) was used at 1:100 dilution. This dilution was made with non-diluted goat anti-mouse IgG1 gold conjugated polyclonal antibody (Arista Biologicals, Inc, PA, USA). The mixture was placed into absorbent paper (GE Health care Life Science), allowed to dry first at 37 degrees for 1 hour followed by room temperature overnight in a desiccant container. Mouse IgG monoclonal antibody (Arista Biologicals, Inc, PA, USA) was applied to the nitrocellulose paper (GE Health care Life Science) and used as a detection standard at a concentration of 11.5 mg/ml.

### HSL Compounds and Conjugates

C12HSL BSA conjugated was acquired from CellMosaic Woburn, Mass., USA and used at full strength. This was applied to nitrocellulose paper (GE Health care Life Science) to detect the present or absent of quorum sensing (QS) molecules. The lack of a signal means that the target analyte is present in the patient sample and competition inhibition has occurred between the mouse monoclonal antibody anti-N-acyl homoserine lactones and the analyte.

N-(3-Oxododecanoyl)-L-homoserine lactone (Chemodex, Switzerland) was used at 8 μM/ml to create a positive control.

Filters, Conjugated and Nitrocellulose Membranes

The sample pads, conjugated pads and nitrocellulose membranes were obtained from GE Health care Life Science. The 0.2 μm filters were purchased from Fisher Scientific, USA.

Buffers Phosphate Buffer Saline (PBS) and Tween-20 were purchased from VWR Life Science, USA. This buffer was used to dilute the extracted patient samples to enhance the motility of the test. Dichloromethane (DCM) (Fisher Scientific, USA) and Methanol (Fisher Scientific, USA) used for the extraction of the analyte from the patient specimen. Acetyl Nitrile (Fisher Scientific, USA) was used to resuspend the analyte after extracted from patient sputum or fluids.

Lateral Flow

A lateral flow immunochromatographic assay was assemble as described above. Briefly, lateral flow is based on a group of capillary beds, such as pieces of permeable paper. Each of these parts has the capacity to move fluid unprompted. The first paper (the sample pad) acts as a sponge and holds an excess of sample fluid. Once saturated, the liquid migrates to the second paper (conjugate pad) in which we have stored the conjugate, a dried format of bio-active particles that contains everything to assure an optimized reaction between the target molecule (an antigen or analyte) and its chemical companion (antibody) that has been immobilized on the particle's surface. Monoclonal mouse IgG1 with the secondary polyclonal goat anti-mouse gold conjugated antibody were added. The sample fluid, 50% PBS-Tween 20 and 50% acetonitrile, dissolves this milieu together with the components of the test simultaneously. The sample and conjugate mix will move through the pervious structure. Here, the analyte binds to the particles while migrating further through the third capillary network. Additionally, the conjugated sample mix reaches these strips, analyte has been bound on the particle and the third capture molecule binds the complex. Finally, when more fluid has passed, the bound particles accumulate and the area changes color. Typically, there are at least two lines. The control line captures any particle and thus shows that reaction has worked properly. The second line contains a specific moiety that only arrests those particles specific for the analyte molecules that have been halted. After migrating thru these reaction areas, the fluid enters the final porous material.

### Results

Lateral Flow performed with PBS-Tween 20 and patient samples to demonstrate the sensitivity of the assay in detecting Pseudomonas aeruginosa using the samples collected from patients. Patient No. 3, 6, 7, 10, 11, and 14 presented clinical manifestation of pneumonia; samples from these patients were tested here. The patient identified as N-2 was a patient infected with bacteria which were not Pseudomonas aeruginosa. 60 μl of PBS-Tween 2 and patient sample were added and allowed to run for 15 minutes. Top band is the C12 BSA conjugate to determine the presence or absence of the QS molecule. The bottom band is the mouse IgG1 to indicate the presence of the sample. One band was clearly observed in all tested patients with Pseudomonas aeruginosa and 2 bands in the patient (Negative 2) infected with different bacteria.

Negative controls and positive controls were used alongside with these patient samples. Lateral Flow was performed with PBS-Tween-20 and C12 HSL was used as a positive control. 30 μl of PBS-Tween 20 were added to 30 μl of C12 HSL (16.8 mg/ml) and the mixture was allowed to run for 15 minutes after loading onto the sample pad. Only one band was observed, i.e., bottom band which is the mouse IgG1 indicating the presence of the sample and the completion of the reaction. Top band which is the C12 BSA conjugate was negative due to competitive inhibition between the analyte (CL12 HSL) and the antibody.

Lateral Flow performed with PBS-Tween 20 to demonstrate a negative control. 60 μl of PBS-Tween 20 were added and allowed to run for 15 minutes.

Two bands were clearly visible. Top band is the C12 BSA conjugate to determine the presence or absence of the QS molecule. The bottom band is the mouse IgG to indicate the presence of the sample.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or will be able to ascertain using no more than routine experimentation many equivalents to the specific embodiments of the invention described herein. Such equivalents are intended to be encompassed by the following claims.

1. A kit for detecting a quorum sensing molecule comprising:
a solid support, wherein a first antibody is immobilized on the solid support; and
a detection agent, wherein the detection agent comprises a detection element,
wherein either (a) the detection agent further comprises a second antibody specific for the quorum sensing molecule and the first antibody is specific for the second antibody or (b) the detection agent further comprises the quorum sensing molecule and the first antibody is specific for the quorum sensing molecule.

2. The kit of claim 1, wherein the solid support is in the form of a test strip.

3. The kit of claim 2, wherein the test strip is an immunochromatographic test strip.

4. The kit of claim 2, wherein the test strip comprises a conjugate pad and a membrane, wherein the membrane comprises a test line and a control line, wherein the test line is closer to the conjugate pad than the control line.

5. The kit of claim 4, wherein the detection agent comprises the detection element and the second antibody, wherein the first antibody is specific for the second antibody, wherein the first antibody is immobilized on the membrane at the control line, wherein the quorum sensing molecule is immobilized on the membrane at the test line, wherein the detection agent is detachably fixed on the conjugate pad.

6. The kit of claim 4, wherein the detection agent comprises the detection element and the quorum sensing molecule, wherein the first antibody is specific for the quorum sensing molecule, wherein the first antibody is immobilized on the membrane at both the control line and the test line, wherein the detection agent is detachably fixed on the conjugate pad.

7. The kit of claim 4, wherein the detection agent comprises the detection element and the second antibody, wherein the first antibody is specific for the second antibody, wherein the first antibody is immobilized on the membrane at the control line, wherein a third antibody specific for the quorum sensing molecule is immobilized on the membrane at the test line, wherein the second antibody and the third antibody bind to the quorum sensing molecule noncompetitively, wherein the detection agent is detachably fixed on the conjugate pad.

8. The kit of claim 7, wherein the membrane further comprises an analyte line, wherein the analyte line is disposed between the test line and the control line, wherein the quorum sensing molecule is immobilized on the membrane at the analyte line.

9. The kit of claim 1, wherein the kit further comprises a reporter agent, wherein the reporter agent can facilitate detection of the detection element.

10. The kit of claim 9, wherein the second antibody, the reporter agent, and the detection element are components of an enzyme-linked immunosorbent assay (ELISA) system.

11. The kit of claim 1, wherein the detection element is an enzyme, wherein the enzyme catalyzes a reaction that can produce a detectable signal.

12. The kit of claim 11, wherein the reporter agent is an enzymatic substrate for the enzyme, wherein the enzyme can act on the reporter agent to produce the detectable signal.

13. A method of detecting the existence of a significant bacterial infection in a subject, the method comprising:
bringing into contact a sample from the subject and the solid support of the kit of claim 1; and
detecting the detection element on the solid support,
wherein the presence or absence of the detection element on the solid support or at a particular location on the solid support indicates the presence of the quorum sensing molecule in the sample, wherein the indication of the presence of the quorum sensing molecule in the sample indicates the existence of a significant bacterial infection in the subject.

14. The method of claim 13, wherein the solid support is in the form of a test strip.

15. The method of claim 14, wherein the test strip is an immunochromatographic test strip.

16. The method of claim 14, wherein the test strip comprises a conjugate pad and a membrane, wherein the membrane comprises a test line and a control line, wherein the test line is closer to the conjugate pad than the control line.

17. The method of claim 16, wherein the detection agent comprises the detection element and the second antibody, wherein the first antibody is specific for the second antibody, wherein the first antibody is immobilized on the membrane at the control line, wherein the quorum sensing molecule is immobilized on the membrane at the test line, wherein the detection agent is detachably fixed on the conjugate pad.

18. The method of claim 17, wherein the absence of the detection element at test line on the solid support indicates the presence of the quorum sensing molecule in the sample.

19. The method of claim 16, wherein the detection agent comprises the detection element and the quorum sensing molecule, wherein the first antibody is specific for the quorum sensing molecule, wherein the first antibody is immobilized on the membrane at both the control line and the test line, wherein the detection agent is detachably fixed on the conjugate pad.

20. The method of claim 19, wherein the absence of the detection element at test line on the solid support indicates the presence of the quorum sensing molecule in the sample.

21. The method of claim 16, wherein the detection agent comprises the detection element and the second antibody, wherein the first antibody is specific for the second antibody, wherein the first antibody is immobilized on the membrane at the control line, wherein a third antibody specific for the quorum sensing molecule is immobilized on the membrane at the test line, wherein the second antibody and the third antibody bind to the quorum sensing molecule noncompetitively, wherein the detection agent is detachably fixed on the conjugate pad.

22. The method of claim 21, wherein the membrane further comprises an analyte line, wherein the analyte line is disposed between the test line and the control line, wherein the quorum sensing molecule is immobilized on the membrane at the analyte line.

23. The method of claim 21, wherein the presence of the detection element at test line on the solid support indicates the presence of the quorum sensing molecule in the sample.
